(19) United States
(12) Patent Application Publication
(10) Pub. No.: US 2011/0263516 A1
(43) Pub. Date: Oct. 27, 2011

(54) VASODILATOR

(75) Inventors: Tatsuhiko HIROTA, Sagamihara-shi (JP); Kohji OHKI, Sagamihara-shi (JP)

(73) Assignee: CALPIS CO., LTD., Tokyo (JP)

(21) Appl. No.: 13/175,340

(22) Filed: Jul. 1, 2011

Related U.S. Application Data
(63) Continuation of application No. 12/279,218, filed on Dec. 1, 2008, now abandoned.

(30) Foreign Application Priority Data
Feb. 14, 2006 (JP) 2006-035944

(51) Int. Cl. A61K 38/06; A61P 9/08

(52) U.S. Cl. 514/21.9

(57) ABSTRACT

There is provided a vasodilator having an endothelium-dependent vasodilator effect, a prophylactic of arteriosclerosis, and functional food having an endothelium-dependent vasodilator effect, which is suitable for daily and regular intake and excellent in safety. The vasodilator and the functional food according to the present invention are characterized in at least one of the tripeptides Val-Pro-Pro and Ile-Pro-Pro, or a proteolytic product containing Val-Pro-Pro and/or Ile-Pro-Pro, contained as an active component.

### CROSS REFERENCE TO RELATED APPLICATIONS

This application is a continuation of U.S. patent application Ser. No. 12/279,218 filed Dec. 1, 2008, which is a National Stage of International Application No. PCT/JP2007/052569 filed Feb. 14, 2007, claiming priority based on Japanese Patent Application No. 2006-035944, filed Feb. 14, 2006, the contents of all of which are incorporated herein by reference in their entirety.

### FIELD OF ART

The present invention relates to a vasodilator having an endothelium-dependent vasodilator effect, and functional food having such an effect.

### BACKGROUND ART

Ischemic diseases, such as myocardial or cerebral infarction, which are the ultimate development of arteriosclerotic diseases, account for major part of the cause of death in Japan, along with cancers. Risk factors for arteriosclerosis include hyperlipemia, hyperlipidemia, hypertension, diabetes, smoking, obesity, hyperuricemia, aging, stress, and the like, which are interrelated to cause angiopathy. Thus even if each risk factor is low, cumulation of the factors additively and synergistically increases the risk, which also increases the risk of ischemic diseases.

On the other hand, it is envisaged that mere mitigation of one of the above risk factors will not present onset of arteriosclerosis. For example, Non-patent Publications 1 and 2 report the absence of interrelationship between the blood cholesterol level and onset of arteriosclerosis, Non-patent Publication 3 teaches that suppression of hypertension does not change the degree of arteriosclerosis, and Non-patent Publication 4 describes that administration of an angiotensin converting enzyme inhibitor containing enalapril as an active component does not result in an arteriosclerosis inhibitory effect. Further, when infarcted, ischemic diseases, for example, acute heart failure is developed, according to Non-patent Publication 5, medicament, such as a vasodilator including nitroprusside or nitroglycerine, a diuretic, or a cardiotonic, is needed to stabilize the hemodynamics.

Thus, though Patent Publications 1 and 2, for example, disclose that tripeptides Val-Pro-Pro and Ile-Pro-Pro have an angiotensin I converting enzyme inhibitory activity, which leads to a hypotensive effect, and also an anti-stress effect, this does not mean that these tripeptides have an anti-arteriosclerotic effect or a vasodilator effect.

Arteriosclerosis is a pathology wherein the arterial wall is thickened to loose its elasticity. One of the factors for such symptom is recently considered to be injury or decreased functions of vascular endothelial cells.

Thus an endothelium-dependent vasodilator is expected to have an inhibitory effect on arteriosclerosis.
Patent Publication 1: JP-6-197786-A
Patent Publication 2: JP-11-100328-A
Non-patent Publication 1: Shoku no Kagaku 257 (1999), p 20-25
Non-patent Publication 2: Atherosclerosis 151 (2000), p 501-508
Non-patent Publication 3: Circulation 104 (2001), p 2391-2394
Non-patent Publication 4: International Journal of Cardiology 81 (2001), p 107-115
Non-patent Publication 5: Bessatsu Igaku no Ayumi, Junkanki Shikkann, state of arts ver. 2 (2001), p 332-334

### SUMMARY OF THE INVENTION

It is an object of the present invention to provide a vasodilator having an endothelium-dependent vasodilator effect.

It is another object of the present invention to provide functional food having an endothelium-dependent vasodilator effect, which may be subjected to daily and regular intake, and is excellent in safety.

According to the present invention, there is provided a vasodilator comprising at least one of peptides Val-Pro-Pro and Ile-Pro-Pro as an active component.

According to the present invention, there is also provided a vasodilator comprising a proteolytic product containing Val-Pro-Pro and/or Ile-Pro-Pro as an active component.

According to the present invention, there is further provided functional food comprising at least one of peptides Val-Pro-Pro and Ile-Pro-Pro as an active component, and having a vasodilator effect.

According to the present invention, there is further provided functional food comprising a proteolytic product containing Val-Pro-Pro and/or Ile-Pro-Pro as an active component, and having a vasodilator effect.

According to the present invention, there is provided use of at least one of peptides Val-Pro-Pro and Ile-Pro-Pro, or use of a proteolytic product containing Val-Pro-Pro and/or Ile-Pro-Pro, in the manufacture of a vasodilator or functional food having a vasodilator effect.

According to the present invention, there is also provided a method of dilating blood vessels comprising the step of administering to an animal an effective amount of at least one of peptides Val-Pro-Pro and Ile-Pro-Pro, or a proteolytic product containing Val-Pro-Pro and/or Ile-Pro-Pro.

Containing, as an active component, Val-Pro-Pro and/or Ile-Pro-Pro derived from animal milk casein or the like, or a proteolytic product containing at least one of these peptides, the vasodilator and the functional food according to the present invention are excellent in safety, and have an endothelium-dependent vasodilator effect. In particular, the functional food may be taken regularly.

The vasodilator according to the present invention is useful for ensuring blood circulation when a patient is suffered from an ischemic disease, such as cardiac or cerebral infarction, relaxes blood vessels which are more prone to constriction due to aging, lifestyle-related diseases or the like, and may be expected to prevent arteriosclerosis, or neck stiffness, cold constitution, thrombosis, or the like symptoms associated with blood flow dysfunction. The functional food according to the present invention is useful as health foods or foods for specified health uses, claiming the vasodilator effect as well as effects on various symptoms or diseases associated with blood flow dysfunction, such as neck stiffness, cold constitution, thrombosis, or the like.

### BRIEF DESCRIPTION OF THE DRAWINGS

FIG. 1 is a graph showing the results of vasodilating tests conducted in Example 1 and Comparative Example 1.

FIG. 2 is a graph showing the results of a confirmatory test for the endothelium dependency of the vasodilator response conducted in Example 1.

### PREFERRED EMBODIMENTS OF THE INVENTION

The present invention will now be explained in detail.

The vasodilator and the functional food according to the present invention contain, as an active component, Val-Pro-Pro and/or Ile-Pro-Pro (these tripeptides are abbreviated as VPP and IPP, respectively, hereinbelow) or a proteolytic product containing at least one of these tripeptides.

The tripeptides may be those to which a pharmacologically acceptable salt has been added, including salts of inorganic acids, such as hydrochlorides, sodium salts, and phosphates, or salts of organic acids, such as citrates, maleates, fumarates, tartrates, and lactates.

The tripeptides may be prepared by digesting and purifying peptides or proteins containing the amino acid sequence VPP and/or IPP through fermentation with microorganisms, by enzymatic hydrolysis of such peptides or proteins, or by synthesis. For detail, see Patent Publications 1 and 2 mentioned above. Thus, the active component of the present invention may be a fermentation product containing at least one of the peptides VPP and IPP obtained by fermentation of peptides or proteins containing the amino acid sequence VPP and/or IPP with microorganisms, or a purified product thereof, or a hydrolysate containing at least one of the peptides VPP and IPP obtained by digesting peptides or proteins containing the amino acid sequence VPP and/or IPP with enzymes, or a purified product thereof.

The effective dose of the vasodilator of the present invention is usually 10 μg to 10 g, preferably about 1 mg to 1 g per day for human in terms of the tripeptides, for achieving the effect in a single dose.

The administration schedule of the vasodilator may be adjusted to the symptoms of a disease. For acute symptoms, single or continuous parenteral administration is suitable. For chronic symptoms or for prophylactic use, regular oral administration for 30 days or longer is preferred.

The administration route of the vasodilator according to the present invention may either be oral or parenteral. The parenteral administration may be topical, transdermal, intravenous, intramuscular, subcutaneous, intradermal, intraperitoneal, intrathoracic, or intraspinal administration. Direct administration to the diseased area is also possible.

The form of the vasodilator according to the present invention may be decided depending on the administration route, and may be in the form of a formulation, such as tablets, pills, hard capsules, soft capsules, microcapsules, powders, granules, liquids, suspensions, or emulsions.

The formulation may be made with, for example, a carrier, adjuvant, excipient, auxiliary excipient, antiseptic, stabilizer, binder, pH regulator, buffer, thickener, gelatinizer, preservative, anti-oxidant, or the like which are acceptable for pharmaceutical use, as desired, in a unit dose form that is required in generally approved formulation.

The functional food according to the present invention may be dispensed as health foods or foods for specified health uses, claiming or advertising the vasodilator effect as well as effects on various diseases associated with blood flow dysfunction.

The amount of intake for obtaining such effect, in particular the vasodilator effect or associated improving effect on a disease, is usually 10 μg to 10 g, preferably about 1 mg to 1 g per day in terms of the tripeptides, taking into account the fact that the present functional food may be taken regularly and daily over a prolonged period of time. The single intake of the functional food may be less than the above amount in terms of the tripeptides, depending on the number of intakes per day.

The period for taking the functional food of the present invention is not particularly limited, and it is preferred to take it for a prolonged period of time for improving chronic symptoms or for prophylactic use. In order to obtain the effect discussed above, regular intake for 30 days or longer, particularly about 3 to 12 months, is preferred.

The functional food according to the present invention may be produced by adding the active component tripeptides, or a food material containing the tripeptides, to various food and beverage, and thus may be made into a variety of forms of food and beverage. For example, the present functional food may be in the form of tablet candies, yogurt, milk beverages, dairy products, alcoholic beverages, refreshing beverages, powdered or granulated food, encapsulated food, various fortified food, or supplements. The functional food of the present invention may optionally contain various additives usually used in food.

### EXAMPLES

The present invention will now be explained in more detail with reference to Examples, which are illustrative only and do not intend to limit the present invention.

### Synthesis Example

IPP and VPP were synthesized through the following organic chemical synthesis by the solid phase method in an automated peptide synthesizer (PSSM-8) manufactured by SHIMADZU CORPORATION.

50 mg of 2-chlorotrityl polystyrene resin to which proline having its amino group protected with a fluorenylmethyloxycarbonyl group (abbreviated as Fmoc hereinbelow) was bound (registered trademark SynProPep Resin, manufactured by SHIMADZU CORPORATION), was used as a solid support. 100 μmol each of Fmoc-Ile, Fmoc-Pro, and Fmoc-Val, wherein the amino groups were protected with the Fmoc group, were sequentially reacted by a routine method according to the amino acid sequence mentioned above to obtain a peptide-bound resin.

The peptide-bound resin was suspended in 1 ml of reaction liquid A (10 vol % acetic acid, 10 vol % trifluoroethanol, and 80 vol % dichloromethane), reacted at room temperature for 30 to 60 minutes to cleave the peptides from the resin, and filtered through a glass filter. The solvent in the resulting filtrate was removed under reduced pressure, and immediately 1 ml of reaction liquid B (82.5 vol % trifluoroacetic acid, 3 vol % ethyl methyl sulfide, 5 vol % purified water, 5 vol % thioanisol, 2.5 vol % ethanedithiol, and 2 vol % thiophenol) was added. The resulting mixture was reacted at room temperature for 6 hours to remove the side chain protective groups, to which 10 ml of anhydrous ether was added to precipitate the peptides. The precipitate was separated by centrifugation at 3000 rpm for 5 minutes, washed several times with anhydrous ether, and dried under nitrogen gas. All of the crude synthesized peptides thus obtained was dissolved in 2 ml of a 0.1 N aqueous solution of hydrochloric acid, and purified by C18 reverse phase HPLC under the following conditions. Pump: model L6200 intelligent pump (manufactured by HITACHI, LTD.); Detector: ultraviolet absorption at 215 nm was detected with model L4000 UV detector (manufactured by HITACHI, LTD.); Column: μBondasphere 5μ C18 (manufactured by Nihon Waters K.K.); Eluate: Liquid A of a 0.1 wt % TFA aqueous solution and Liquid B of 0.1 wt % TFA-containing acetonitrile, (B/A+B)×100(%): 0 to 40% (over 60 min); Flow rate: 1 ml/min.

The eluted fraction having the maximum absorption was taken out and lyophilized to obtain the objective synthesized peptides Ile-Pro-Pro and Val-Pro-Pro at the yields of 5.7 mg and 6.5 mg, respectively. The purified peptides were analyzed from the N-terminal in an automated protein sequencer (model PPSQ-10, manufactured by SHIMADZU CORPORATION), and further analyzed in an amino acid analyzer (model 800 series, manufactured by JASCO CORPORATION). It was confirmed that the peptides were prepared as designed.

### Example 1

### <Vasodilating Test>

The thoracic aorta of a Wistar rat was taken out, cut into 2 mm long, and made into an aorta ring. The ring was set in a Magnus apparatus (product name “micro tissue organ bath MTB-1Z”, manufactured by LABO SUPPORT CO., LTD.), and allowed to equilibrate with a constant tension. The constriction response of the aorta ring was confirmed with 50 mM KCl. The aorta ring was then allowed to constrict with 1 μM of phenylephrine, and the stably constricted samples were observed for endothelium-dependent vasodilator response using 1 μM of acetylcholine to confirm that the endothelial functions were maintained.

Next, the aorta ring was preliminarily constricted with 1 μM phenylephrine, and VPP and IPP prepared in Synthesis Example were added at ten-fold increasing concentrations from 10⁻⁹ M to observe the vasodilator response through the change in tension of the aorta ring. The dose dependency of the vasodilator response was also studied.

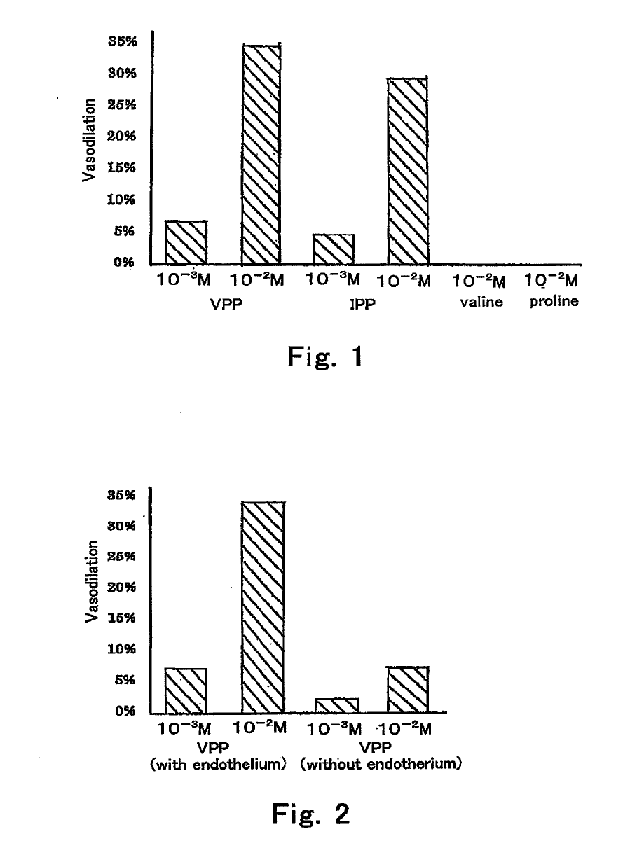

The result was that the vasodilator response to VPP and IPP was observed from the concentration of 1 mM, and a clear, transient vasodilator response was confirmed at 10 mM. The results are shown in FIG. 1.

### <Confirmatory Test for Endothelium Dependency of Vasodilator Response to VPP and IPP>

In order to confirm that the vasodilator effect of VPP and IPP was associated with the vascular endothelium, a vasodilating test similar to the above was conducted with VPP, using as a control a blood vessel from which the vascular endothelium was physically removed by a routine method. The result was that the vasodilator response was weakened due to the removal of the endothelium, indicating that the vasodilating effect of VPP and IPP was highly endothelium dependent. The results are shown in FIG. 2.

### Comparative Example 1

The vasodilating test was conducted in the same way as in Example 1, except that the tripeptides VPP and IPP were replaced with amino acids, valine (Val) and proline (Pro), at the same concentration.

The results were that no vasodilator response was observed at 10 mM with either Val or Pro. The results are shown in FIG. 1.

From the results discussed above, it is understood that VPP and IPP are active as vasodilators in the peptide forms.

### Referential Example

JP-2004-244359-A reports a vasodilating medical composition and a vasodilating health foods composition containing, as an active component, peptides obtained by hydrolyzing proteins derived from various milk proteins. Based on the conventional knowledge that β-casein and K-casein have the amino acid sequences including VPP and IPP, the peptides obtained in the Production Examples disclosed in this publication were measured for VPP and IPP.

Commercially available skim milk, which is a protein material containing β-casein and κ-casein was tested among the material used in the Production Examples. 1 kg of the skim milk was suspended in 2 L of warm water, adjusted to pH 7.5, mixed with 40 g of thermoase (manufactured by DAIWA FINE CHEMICALS CO., LTD.), and reacted at 50° C. for 16 hours. After the reaction, the reaction liquid was heated at 100° C. for 10 minutes for inactivating the enzyme, to obtain hydrolyzed peptides. Through the analysis with a high performance liquid chromatograph-mass spectrometer, it was confirmed that the obtained hydrolyzed peptides did not include VPP or IPP.

From the results discussed above, it was demonstrated that the vasodilation reported in JP-2004-244359-A was caused by components other than VPP and IPP.

What is claimed is:

1. A method for dilating blood vessels comprising administering to an animal an effective amount of at least one of peptides Val-Pro-Pro and Ile-Pro-Pro.

2. A method for dilating blood vessels comprising administering to an animal an effective amount of a proteolytic product containing Val-Pro-Pro and/or Ile-Pro-Pro.

3. The method according to claim 1, wherein said effective amount is 10 μg to 10 g in terms of the tripeptides.

4. The method according to claim 1, wherein said effective amount is administered parenterally.

5. The method according to claim 4, wherein said effective amount is administered in single or continuous parenteral administration for acute symptoms.

6. The method according to claim 1, wherein said effective amount is administered orally.

7. The method according to claim 6, wherein said effective amount is administered in regular oral administration for 30 days or longer for chronic symptoms or for prophylactic use.

8. The method according to claim 1, wherein said blood vessels are dilated endothelium-dependently.

9. The method according to claim 2, wherein said effective amount is 10 μg to 10 g in terms of the tripeptides.

10. The method according to claim 2, wherein said effective amount is administered parenterally.

11. The method according to claim 10, wherein said effective amount is administered in single or continuous parenteral administration for acute symptoms.

12. The method according to claim 2, wherein said effective amount is administered orally.

13. The method according to claim 12, wherein said effective amount is administered in regular oral administration for 10 days or longer for chronic symptoms or for prophylactic use.

14. The method according to claim 2, wherein said blood vessels are dilated endothelium-dependently.
